(19) United States
(12) Patent Application Publication
(10) Pub. No.: US 2014/0030296 A1
(43) Pub. Date: Jan. 30, 2014

(54) VACCINE ADJUVANT

(71) Applicants: SHIZUOKA PREFECTURE, Shizuoka (JP); OLYMPUS CORPORATION, Tokyo (JP)

(72) Inventors: Kouji MARUYAMA, Shizuoka (JP); Mitsuru TAKAHASHI, Shizuoka (JP); Masahiro NAKAGAWA, Shizuoka (JP); Yasuto AKIYAMA, Shizuoka (JP); Hidee ISHII, Shizuoka (JP); Jinyan CHENG, Tokyo (JP)

(73) Assignees: SHIZUOKA PREFECTURE, Shizuoka (JP); OLYMPUS CORPORATION, Tokyo (JP)

(21) Appl. No.: 13/948,273

(22) Filed: Jul. 23, 2013

Related U.S. Application Data
(63) Continuation of application No. PCT/JP2012/000584, filed on Jan. 30, 2012.

(30) Foreign Application Priority Data
Jan. 31, 2011 (JP) JP 2011-018844

(51) Int. Cl. A61K 33/42; A61K 39/00

(52) U.S. Cl. CPC A61K 33/42; A61K 39/0011
USPC 424/400; 424/277.1

(57) ABSTRACT

A method for enhancing a vaccine therapy effect by a vaccine composition. The method including a step of implanting β-tricalcium phosphate in an inside of a body of a subject, and a step of administering the vaccine composition to the subject at the same time with or before or after the implanting step.

### TECHNICAL FIELD

The present invention relates to an agent for enhancing vaccine therapy effect (vaccine adjuvant) and a vaccine therapy kit, and more particularly, to an agent for enhancing vaccine therapy effect using a vaccine composition, comprising β-tricalcium phosphate (TCP) to be implanted for use inside of a body of a subject, as an active ingredient, and a vaccine therapy kit consisting of a composition comprising β-tricalcium phosphate and a vaccine composition.

### BACKGROUND ART

Immunity is a defense system of the living body, by which bacteria and viruses that have invaded exogenously through the surface barrier into the body are eliminated. The immune response is induced and regulated in a complex manner by B lymphocytes, T lymphocytes, antibodies, antigen-presenting cells (APC), and so on. First of all, foreign antigens that have been taken up by and processed in APC are bound to the major histocompatibility (MHC) class I and class II molecules and presented to helper T cells. Upon recognition of MHC-bound foreign antigens by helper T cells, activation of T cells takes place. The activated T cells secrete cytokines to help antigen-stimulated B cells to differentiate into antibody-producing cells, while promoting differentiation of killer T cells. Cells representing the antigen are eliminated by the antibodies secreted by B cells and by the activated T cells, whereby the cell-mediated and humoral reactions for eliminating foreign antigens proceed.

Furthermore, immunity acts not only against pathogens invading exogenously, but also against abnormal autologous cells such as cells that have turned cancerous or cells that have been infected with viruses or bacteria, which are originally autologous cells. Recently, expectations have been raised for vaccine therapy that utilizes the inherent immune system in the living body as described above to treat or prevent diseases, in which immunity against abnormal cells is induced by artificial administering, as antigens, the components of abnormal cells that can be recognized by the host's immune system as the target antigen. In the conventional cancer therapy, surgical operations and radiotherapy are the primary treatment, but there are problems in that minute fragments of cancer tissue are left behind in the body, which cause cancer recurrence. In light of this, eradication of minute fragments of cancer tissue that may be left behind in the body and prevention of recurrence are expected with vaccine therapy.

As the vaccine antigen used for vaccine therapy, for example, a vaccine TLP peptide to be used for the prevention or treatment of tumor (Patent Document 1), a method of identifying specific tumor antigens by means of the selection of cDNA display libraries using sera and tumor antigens thus identified (Patent Document 2), and a vaccine comprising a polypeptide comprising an MHC Class I-binding epitope of mesothelin (Patent Document 3) are disclosed. Up until today, research has been attempted on vaccine therapy with various tumor antigens. However, the current situation is that with respect to the therapeutic effect on cancer patients, none of the vaccine therapies is as fully effective as expected. An adjuvant is also called an antigenicity-enhancing agent, which is a reagent used to potentiate the antigenicity. In light of this, there has been a demand for a supplementary agent for vaccine therapy that potentiates the antigenicity to achieve a full effect of vaccine therapy, i.e., adjuvant.

Meanwhile, by virtue of the excellent biocompatibility of a calcium phosphate compound comprising β-tricalcium phosphate, applied research of this compound as a biomaterial such as an artificial bone and artificial tooth root is actively ongoing (Patent Documents 4 and 5). The present inventors revealed that porous β-tricalcium phosphate suppressed malignant tumor by activating the macrophage activity, and developed a cancer cell inhibitor consisting of a cancer cell inhibiting sheet, which is a sheet coated with a powder of β-tricalcium phosphate having a porous rate of 75% (Patent Document 6, Non-patent Document 1). However, the effect of the combined use of β-tricalcium phosphate and a vaccine composition has been entirely unknown.

Also, there have been some attempts to construct artificial lymph nodes; however, effort to develop such an immune device that has a highly similar structure to native lymph nodes and actually exerts a potent immune function in vivo has been entirely in vain. For example, Patent Document 7 discloses that when feeder cells (stromal cells) are seeded on a support prepared using a collagen sponge and the resulting support is implanted under the renal capsule, the support serves as an artificial lymph node and potently induces immune reactions such as antibody production and cancer cell elimination. However, all of these techniques use cells, and therefore have required the steps of in vitro cell culture and construction using artificial materials.

### PRIOR ART DOCUMENTS

### Patent Documents

Patent Document 1: Japanese unexamined Patent Application Publication (Translation of PCT Application) No. 2003-523759
Patent Document 2: Japanese unexamined Patent Application Publication (Translation of PCT Application) No. 2005-508309
Patent Document 3: Japanese unexamined Patent Application Publication (Translation of PCT Application) No. 2006-521090
Patent Document 4: Japanese unexamined Patent Application Publication No. 2010-233914
Patent Document 5: Japanese unexamined Patent Application Publication No. 7-116175
Patent Document 6: Japanese unexamined Patent Application Publication No. 2010-126434
Patent Document 7: Japanese unexamined Patent Application Publication No. 2008-163015

### Non-Patent Document

Non-patent Document 1: J. Cheng et al., J. Biomed. Mater. Res. A, 92(2): 542-547 (2010)

### SUMMARY OF THE INVENTION

### Object to be Solved by the Invention

Recently, expectations have been raised for vaccine therapy that utilizes the inherent immune system in the living body to treat or prevent a disease, in which immunity against abnormal cells is induced by artificial administering, as antigens, the components of abnormal cells that can be recognized by the host's immune system as the target antigen. However, the current situation is that with respect to, for example, the therapeutic effect on cancer patients, the existing vaccine therapy is not as fully effective as expected. In order to solve this problem, an inexpensive, safe supplementary agent for vaccine therapy that potentiates the antigenicity to achieve a full effect of vaccine therapy is demanded. An object of the present invention is to provide an inexpensive, safe adjuvant capable of potentiating the effect of a vaccine composition.

### Means to Solve the Object

The present inventors found that lymphocytes such as T cells, B cells, and NK cells and dendritic cells that are activated, induced, and accumulated by a β-tricalcium phosphate dense body (referring to ones having a porous rate of 50% or less here) implanted in the vicinity of a lesion formed a highly organized three-dimensional structure. By utilizing this, lymphocytes initiate antigen-specific immune response (adoptive immunity) through interaction with the antigens and antigen-presenting cells, whereby the therapeutic effect of a vaccine composition on cancer can be potentiated.

That is, the present invention relates to [1] a vaccine therapy kit, comprising: a composition comprising β-tricalcium phosphate to be implanted for use inside of a body of a subject as an active ingredient; and a vaccine composition, [2] the vaccine therapy kit according to [1], wherein the inside of a body is in a skin, under a skin, or in a muscle, [3] the vaccine therapy kit according to [1] or [2], wherein the β-tricalcium phosphate is to be implanted in a vicinity of a lesion, [4] the vaccine therapy kit according to [3], wherein the vicinity of a lesion is an area at a distance of 0.1 to 15 cm from the lesion, [5] the vaccine therapy kit according to any one of [1] to [4], wherein the vaccine composition is a cancer-specific antigen vaccine, and [6] the vaccine therapy kit according to any one of [1] to [5], wherein the cancer-specific antigen vaccine is an antigenic peptide or protein derived from tyrosinase-related protein (TRP-2), Wilms tumor 1 (WT1), ovalbumin (OVA), or a tumor lysate.

The present invention also relates to [7] the vaccine therapy kit according to any one of [1] to [6], wherein the β-tricalcium phosphate is in a form of a dense body having a porous rate of 50% or less, [8] the vaccine therapy kit according to [7], wherein the β-tricalcium phosphate activates, induces, or accumulates T cells, B cells, NK cells, dendritic cells, and macrophages, [9] the vaccine therapy kit according to any one of [1] to [8], wherein the β-tricalcium phosphate is in a form of a tablet or a columnar body, and [10] the vaccine therapy kit according to any one of [1] to [8], wherein the β-tricalcium phosphate is in a form of a particle or granule having a particle diameter of 0.05 to 25 μm.

The present invention also relates to [11] an agent for enhancing vaccine therapy effect with a vaccine composition, comprising β-tricalcium phosphate to be implanted for use inside of a body of a subject as an active ingredient, [12] the agent for enhancing vaccine therapy effect according to [11], wherein the inside of a body is in a skin, under a skin, or in a muscle, [13] the agent for enhancing vaccine therapy effect according to [11] or [12], wherein the β-tricalcium phosphate is implanted in a vicinity of a lesion, [14] the agent for enhancing vaccine therapy effect according to [13], wherein the vicinity of a lesion is an area at a distance of 0.1 to 15 cm from the lesion, [15] the agent for enhancing vaccine therapy effect according to any one of [11] to [14], wherein the vaccine composition is a cancer-specific antigen vaccine, and [16] the agent for enhancing vaccine therapy effect according to [15], wherein the cancer-specific antigen vaccine is an antigenic peptide or protein derived from tyrosinase-related protein 2 (TRP-2), Wilms tumor 1 (WT1), ovalbumin (OVA), or a tumor lysate.

The present invention also relates to [17] the agent for enhancing vaccine therapy effect according to any one of [11] to [16], wherein the β-tricalcium phosphate is in a form of a dense body having a porous rate of 50%, [18] the agent for enhancing vaccine therapy effect for vaccine therapy according to [17], wherein the β-tricalcium phosphate activates, induces, or accumulates T cells, B cells, NK cells, dendritic cells, and macrophages, [19] the agent for enhancing vaccine therapy effect according to any one of [11] to [18], wherein the β-tricalcium phosphate is in a form of a tablet or a columnar body, and [20] the agent for enhancing vaccine therapy effect according to any one of [11] to [18], wherein the β-tricalcium phosphate is in a form of a particle or granule having a particle diameter of 0.05 to 25 μm.

### Effect of the Invention

The present invention can provide a vaccine therapy kit consisting of a composition comprising β-tricalcium phosphate (TCP) to be implanted for use in an inside of a body of a subject as the active ingredient, and a vaccine composition. The present invention can also provide a agent for enhancing vaccine therapy effect comprising β-tricalcium phosphate to be implanted for use inside of a body of a subject as an active ingredient. Therefore, the present invention can potentiate the effect of vaccine therapy to treat diseases.

### BRIEF DESCRIPTION OF DRAWINGS

FIG. 1 shows the result of the scanning electron microscopic observation of the binding of an immune cell to a β-TCP granule (25 to 75 μm in size).

FIG. 2 shows the result of the scanning electron microscopic observation of a β-TCP dense body (a dense body tablet: diameter 5×2 mm). Particles having a diameter of 2 μm are used as markers.

FIG. 3 shows the result of the scanning electron microscopic observation of a β-TCP dense body (a dense body bar: diameter 0.9×5 mm).

FIG. 4 A β-TCP dense body (tablet) was subcutaneously implanted in a normal mouse, and 2 weeks later, a tissue sample was stained with HE and observed under a microscope. FIG. 4 is a diagram showing that the accumulated lymphocytes are configuring a similar structure to a native lymph node.

FIG. 5 A 0.05 to 105 μm-sized β-TCP dense body (granule) was subcutaneously implanted in a normal mouse, and 2 weeks later, a tissue sample was stained with HE and observed under a microscope. FIG. 5 is a diagram showing that 75 μm or larger-sized β-TCP granules induce macrophages (N=13), 75 μm or smaller-sized granules gather lymphocytes (N=11), and 0.05 to 25 μm-sized microgranules form lymphocyte aggregates (arrows in the Figure).

FIG. 6 In a tissue sample of a normal mouse that received subcutaneous implantation of a discoid β-TCP dense body tablet having a diameter of 5×2 mm, B cells were immunohistochemically stained with an anti-CD45R antibody, followed by microscopic observation. FIG. 6 is a diagram showing induction of B cells by the β-TCP dense body (left: low-power ×4, right: high-power ×40, indicated by arrows).

FIG. 7 In a tissue sample of a normal mouse that received subcutaneous implantation of a discoid β-TCP dense body tablet having a diameter of 5×2 mm, T cells were immunohistochemically stained with an anti-CD3 antibody, followed by microscopic observation. FIG. 7 is a diagram showing induction of T cells by the β-TCP dense body (left: low-power ×4, right: high-power ×40, indicated by arrows).

FIG. 8 In a tissue sample of a normal mouse that received subcutaneous implantation of a discoid β-TCP dense body tablet having a diameter of 5×2 mm, NK cells were immunohistochemically stained with an anti-CD49b antibody, followed by microscopic observation. FIG. 8 is a diagram showing induction of NK cells by the β-TCP dense body (left: low-power ×4, right: high-power ×40, indicated by arrows).

FIG. 9 In a tissue sample of a normal mouse that received subcutaneous implantation of a discoid β-TCP dense body tablet having a diameter of 5×2 mm, dendritic cells were immunohistochemically stained with an anti-CD11c antibody, followed by microscopic observation. FIG. 9 is a diagram showing induction of dendritic cells by the β-TCP dense body (left: low-power ×4, right: high-power ×40, indicated by arrows).

FIG. 10 In a tissue sample of a normal mouse that received subcutaneous implantation of a discoid β-TCP dense body tablet having a diameter of 5×2 mm, macrophages were immunohistochemically stained with an anti-F4/80R antibody, followed by microscopic observation. FIG. 10 is a diagram showing induction of macrophages by the β-TCP dense body (left: low-power ×4, right: high-power ×40, indicated by arrows).

FIG. 11 In a tissue sample of a normal mouse that received subcutaneous implantation of a discoid β-TCP dense body tablet having a diameter of 5×2 mm, lymphatic endothelial cells were immunohistochemically stained with an anti-D2-40 antibody, followed by microscopic observation. FIG. 11 is a diagram showing induction of lymphatic vessel formation by the β-TCP dense body (left: low-power ×4, right: high-power ×40, indicated by arrows).

FIG. 12 As an experimental system of vaccines, in a model experimental system of tumor using a C57BL/6 strain mouse and E.G7-OVA (purchased from ATCC), which is the mouse lymphoma cell expressing avian ovalbumin, the following 3 groups were set up: 1) control group, 2) OVA-administered group, and 3) OVA+TCP group. A discoid β-TCP tablet having a diameter of 5 mm×thickness of 2 mm was subcutaneously implanted in the flank of a 6-week-old male mouse (C57BL/6) (OVA+TCP group). Seven and 14 days later, 100 μg of OVA protein was administered intradermally around the implanted β-TCP tablet (OVA+TCP group) or intradermally in the flank (OVA-administered group). Twenty one days later, 1×10⁵ E.G7-OVA cells were subcutaneously transplanted in the flank, and formation and proliferation of tumor was observed. In the Figure, the asterisk * indicates that tumor proliferation was significantly suppressed in the OVA+TCP group, which was immunized with OVA protein after implantation of β-TCP, on 42 days and 45 days after the initiation of the experiment in comparison with the control group (t test, p<0.05).

### MODE OF CARRYING OUT THE INVENTION

The vaccine therapy kit of the present invention is not particularly limited as long as it includes a composition comprising β-tricalcium phosphate (hereinbelow, referred to as “β-TCP”) to be implanted for use in an inside of a body, in other words, for implanting in an inside of a body as the active ingredient, and a vaccine composition. Also, the agent for enhancing vaccine therapy effect using the vaccine composition according to the present invention is not particularly limited as long as it is a composition comprising β-TCP as the active ingredient. One embodiment of the present invention can include a vaccine therapy in which a composition comprising β-tricalcium phosphate as the active ingredient is implanted in an inside of a body of a subject, followed by administration of a vaccine composition. Another embodiment of the present invention can include the use of β-tricalcium phosphate to be implanted for use in an inside of a body of a subject for the production of the agent for enhancing vaccine therapy effect using the vaccine composition. The aforementioned β-TCP is one crystal form of a composition represented by Ca₃(PO₄)₂, which is a biocompatible compound that is stable at normal temperature. As the β-TCP, commercially available β-TCP powders and β-TCP blocks, for example, β-TCP blocks to be used as artificial bone may be used, or the β-TCP can be produced by a publicly known method (Japanese unexamined Patent Application Publication No. 2004-26648, etc.) or from an α-TCP powder (Japanese unexamined Patent Application Publication No. 2006-89311, etc.). Also, while the β-TCP composition of the present invention may be a β-TCP composition substantially composed of β-TCP or a β-TCP composition produced by mixing in a surfactant, water and so on, the β-TCP composition may be a β-TCP powder obtained by processing a β-TCP block into particles or granules, and besides that, the β-TCP composition is preferably a polycrystalline, high-strength and high-density β-TCP dense body having a low porous rate obtained by densificating a β-TCP powder by tablet pressing or after defoaming by sintering, and among such β-TCP dense bodies, one having a porous rate of 50% or less is preferable. In the present invention, a β-TCP dense body refers to β-TCP having a porous rate of 50% or less. Also in the present invention, powders, particles, and granules all indicate the granular form, and there is no substantial distinction among them.

The particle size of the polycrystal composing the β-TCP dense body is preferably 0.001 to 50 μm, more preferably 0.01 to 20 μm, and even more preferably 0.75 to 10.0 μm, and an average particle size of the polycrystal is preferably 1.80 μm. Further, the particles of the polycrystal are spaced apart preferably by 0.001 to 10 μm, more preferably by 0.005 to 5 μm, and by 0.01 to 1.0 μm, and on average, the particles of the polycrystal are spaced apart preferably by 0.05 to 0.2 μm, and more preferably by 0.1 μm. Pores in the β-TCP dense body are determined according to the size and number of spaces between the particles of the polycrystal.

The porous rate of β-TCP can be measured by a mercury intrusion method, in which pressure is applied to force mercury into a pore and the specific surface area and pore distribution are obtained from the pressure applied and the amount of mercury intruded, or from the density using a calibration curve. It is also possible to measure the true density ρ of a β-TCP stone having a porous rate of 0% (a β-TCP sintered body having the same composition as the sintered body of an object to be measured but having no pores in the crystalline form) in advance, and from an apparent density ρ′ calculated from the volume and weight of the sintered body of an object to be measured, calculate the porous rate P(%) as P(%)=(1−ρ′/ρ)×100. Further, the porous rate P can also be measured using, for example, the ACCUPYC 1330 series (the product of Shimadzu Corporation). The porous rate in the β-TCP block as measured by the mercury intrusion method is preferably 50 to 90%, more preferably 60 to 85%, and even more preferably 70 to 80%. A β-TCP powder having a particle diameter of 75 to 105 μm can be produced by pulverizing a β-TCP block, and the porous rate of each powder thus produced as measured by the mercury intrusion method is preferably 50 to 90%, more preferably 55 to 85%, and even more preferably 57 to 80%, and a preferable average porous rate is 60 to 70%. Also, a β-TCP microgranule having a particle diameter of 25 μm or less can also be produced by pulverizing a β-TCP block into particles. The porous rate of the β-TCP microgranule thus produced as measured by the mercury intrusion method is preferably 50% or less, more preferably 40% or less, and even more preferably 30% or less, and a preferable average porous rate is 3.5 to 4.5%. Examples of the β-TCP dense body can include a β-TCP powder, particle, granule, tablet, and columnar body having a porous rate of 50% or less. These β-TCP dense bodies may consist exclusively of β-TCP or comprise β-TCP as the active ingredient as well as other components. The porous rate of β-TCP having a porous rate of 50% or less is preferably 40% or less, more preferably 30% or less, and within the above range, a porous rate of 20% or less is particularly preferable. The porous rate of the β-TCP dense body that is densificated by tablet pressing a β-TCP powder is preferably 50% or less, more preferably 40% or less, and even more preferably 30% or less as calculated from the density, and within the above range, a porous rate of 20% or less can be given as a preferable example. Also, the porous rate of the β-TCP dense body that is densificated after defoaming by sintering is preferably 50% or less, more preferably 40% or less, and even more preferably 30% or less as calculated from the density, and within the above range, a porous rate of 20% or less can be given as a preferable example.

For example, densification by sintering is performed by sintering preferably at above 600° C. to 2000° C., more preferably at 750 to 1500° C., and even more preferably at 900 to 1300° C., and preferably for 1 to 100 hours, more preferably for 10 to 80 hours, and even more preferably for 20 to 50 hours. Further, sintering can be divided into multiple operations and performed at a plurality of different temperatures. For example, a rod-shaped β-TCP dense body can also be produced by the following method. A slurry is prepared by mixing calcium hydrogen phosphate, calcium carbonate, and water at an appropriate ratio, and the slurry thus prepared is allowed to undergo reaction while grinding, followed by drying. Subsequently, the solid material obtained after drying is pulverized and calcined to give a β-TCP powder. The β-TCP powder thus obtained is formed into a columnar body by compression forming. The resulting compression-formed columnar body is sintered at 600° C. to 1300° C. for 20 hours.

Examples of a method for producing the aforementioned β-TCP powder, particle, or granule, which is a dense body having a porous rate of 50% or less, include the following method. That is, a calcium hydrogen phosphate powder and a calcium carbonate powder are weighed out so as to achieve a mole ratio of 1:1 to 3, preferably 1:1.5 to 2.5, and mixed. Then, purified water is added to the resulting calcium hydrogen phosphate-calcium carbonate mixture to prepare a slurry, and the resulting slurry is subjected to wet grinding using a ball mill for approximately 24 hours to carry out reactions by a mechanochemical method. The ground slurry is dried at 70 to 90° C., preferably at 75 to 85° C., and a β-TCP powder obtained by pulverizing the solid obtained after drying is calcined at 700 to 800° C. for several hours, whereby a calcined β-TCP powder is produced. The calcined β-TCP powder thus obtained is sieved to obtain fractions of respective particle diameters, and by measuring the porous rate in each fraction, a fraction having a porous rate of 50% or less can be obtained. Specifically, when it is so defined that firstly β-TCP that has passed through a sieve with a mesh size of 105 μm but did not pass through a sieve with a mesh size of 75 μm is a fraction (A), secondary β-TCP that has passed through a sieve with a mesh size of 75 μm but did not pass through a sieve with a mesh size of 25 μm is a fraction (B), and further β-TCP that has passed through a sieve with a mesh size of 25 μm is a fraction (C), the fraction (C) comprises β-TCP having a porous rate of definitely 50% or less.

Examples of a method for producing the aforementioned β-TCP tablet or β-TCP columnar body include a method capable of producing a β-TCP tablet or columnar body having a porous rate of 50% or less and having a single phase or a near single phase. Specific examples include a method for obtaining a β-TCP tablet or columnar body as a sintered body, including producing a calcined β-TCP powder as described above, forming the calcined β-TCP powder thus obtained into a tablet or columnar body by compression molding (tablet pressing), and sintering the resulting formed, calcined β-TCP tablet or columnar body at 450 to 650° C., preferably at 600° C. for 2.5 to 3.5 hours, preferably for 3 hours, and then at 850 to 950° C., preferably at 900° C. for 0.5 to 1.5 hours, preferably for 1 hour, and then at 1000 to 1300° C. for 0.75 to 1.5 hours, preferably for 1 hour. Further, it is also possible to produce a β-TCP tablet or columnar body by sintering the aforementioned calcined β-TCP powder to obtain a powder as a β-TCP sintered body, and then subjecting the powder to compression molding (tablet pressing). It is also possible to produce a β-TCP columnar body by punching out a β-TCP tablet in a columnar shape.

The β-TCP composition of the present invention may comprise, in addition to β-TCP, a variety of ingredients for drug formulation such as a pharmaceutically acceptable common carrier, a binder, a stabilizer, an excipient, a diluent, a pH buffer, a disintegrant, a solubilizing agent, a solubilizing aid, and an isotonic agent. The β-TCP composition can be provided as a gel, a paste, a suspension of fine particles, and a solid preparation, and it is preferably a solid. Examples of the form of the solid include a granule, a powder, a particle, and also, a discoid tablet (short length circular cylinder) and a rod-shaped tablet (elliptic cylinder).

Also, regarding the particle diameter of a solid β-TCP composition, for example, the particle diameter of a β-TCP powder obtained by processing a β-TCP block into particles is preferably 50 to 120 μm, more preferably 60 to 110 μm, and even more preferably 75 to 105 μm. Also, the particle diameter of a β-TCP microgranule obtained by processing a β-TCP block into particles is preferably 50 μm or less, more preferably 40 μm or less, and even more preferably 30 μm or less. Within the above range, preferable examples include a β-TCP microgranule having a particle diameter of 0.05 to 5 μm and a β-TCP microgranule having a particle diameter of 0.05 to 25 μm. In the present invention, a particle having a particle diameter of 0.05 to 25 μm indicates a particle that has passed through a sieve with a mesh size of 25 μm. With regard to the size of a discoid, columnar, or rod-shaped tablet of the β-TCP composition, the diameter is preferably 0.01 to mm, more preferably 0.1 to 20 mm, and even more preferably 1 to 10 mm, and the length is preferably 0.1 to mm, more preferably 0.5 to 30 mm, and even more preferably 1 to 20 mm. Within the above range, a discoid tablet having a diameter of 5 mm and a length of 2 mm and a columnar bar having a diameter of 0.9 mm and a length of 10 mm can be given as preferable examples.

The inside of a body as used herein is not particularly limited as long as it is a site other than teeth and bone. Examples of such a site include under a skin, in a skin, in a muscle, in a abdominal cavity, in a chest cavity, and in a brain. Under a skin is preferable, and examples include in the vicinity of the lesion to be treated. When the β-TCP of the present invention is implanted for use in the vicinity of a lesion, it can be implanted for use in an area at a distance of preferably 1 μm to 30 cm, more preferably 100 μm to 20 cm, and even more preferably 1 mm to 15 cm from the lesion. The lesion to be treated according to the present invention is not particularly limited as long as it is not a lesion affecting bone or teeth, and examples thereof include cancer, allergic immunity, and infection. Among them, cancer is a preferable example, and particularly preferable examples include cancer of the tissues inside the skull, lung, squamous epithelial cells, skin, soft tissues, bladder, stomach, pancreas, head, neck, kidney, prostate gland, large intestine, small intestine, esophagus, female genitalia (such as ovaries and uterus), or thyroid gland.

The method of administering the β-TCP of the present invention is not particularly limited as long as it is a method of directly implanting the β-TCP into the body by surgical operation. It is also possible to implant the β-TCP of the present invention into an incision site made during surgery performed for other purposes. Also from the viewpoint of reducing a burden imposed on the living body, a method of implanting the β-TCP composition of the present invention from the minimum opening using a common syringe or a syringe for inserting a solid material can be presented as a preferable example. In such a case, examples of the β-TCP composition of the present invention can include a suspension comprising β-TCP granules, β-TCP prepared as a gel or paste, a syringe filled with solid β-TCP such as a device (introduction device) mounted with a rod-shaped (elliptic cylindrical) β-TCP dense body and a set of a rod-shaped (elliptic cylindrical) β-TCP dense body and a device (introduction device) therefor. Also, the dose can be appropriately selected according to the type of the disease, the size of the lesion, the body weight of the patient, and so on, and for example, the dose is preferably 0.01 mg to 100 g, more preferably 0.1 mg to 10 g, and even more preferably 1 mg to 1 g.

The vaccine composition according to the present invention is not particularly limited as long as it is a composition comprising vaccine. The vaccine composition is preferably a composition comprising an antigenic peptide specifically expressed in cancer cells or a composition comprising an antigenic peptide that is expressed at a low level in normal cells but at a high level in cancer cells. It is also possible to use a cancer cell extract, an artificially synthesized antigenic peptide, or an antigenic peptide produced using genetic engineering technique, or it is also possible to obtain a commercially available product and use it. Examples include an already marketed vaccine or a vaccine in the process of development such as Cervarix (GlaxoSmithKline plc) and Gardasil (Merck & Co., under clinical trial), which are vaccines against papillomavirus for the prevention of uterine cervix cancer, and Stimuvax (Merck Serono, under clinical trial), which is a vaccine against mucin 1 (MUC-1) for the treatment of non-small cell lung cancer, and also, a melanoma-associated antigen (MAGE) expressed in melanoma, glycoprotein 100 (gp100), tyrosinase-related protein 2 (TRP-2), a tumor-associated antigen such as cancer/testis antigen 1B (NY-ESO-1) and a carcinoembryonic antigen (CEA), which are expressed in various types of cancer, and also, a group of genes specifically expressed in tumor such as up-regulated lung cancer 10 (URLC10), which has been recently identified, TTK protein kinase (TTK), IGF II mRNA binding protein 3 (KOC1), ring finger protein 43 (RNF43), translocase of outer mitochondrial membrane 34 (TOMM34), cell division cycle associated 1 (CDCA1), kinesin family member 20A (KIF20A), DEP domain-containing protein 1 (DEPDC1), and M phase phosphoprotein 1 (MPHOSPH1), a cancer gene such as an epidermal growth factor receptor 2 (HER2/neu) and Wilms tumor 1 (WT1), a protein that is a product of a cancer suppressor gene such as p53 and its derivative, a growth factor receptor protein such as a vascular endothelial growth factor receptor-1 (VEGFR1) and a vascular endothelial growth factor receptor-2 (VEGFR2), which are associated with angiogenesis extending to tumor, and a pharmaceutical composition comprising an antigenic peptide derived from a protein expressed specifically or preferentially in tumor or peritumoral tissues (VEGFR1 and VEGFR2). Also, the length of the above antigenic peptide can be appropriately prepared from one amino acid to the full length of protein, and the above antigenic peptide may be preferably from 1 to 1000 amino acid long, more preferably 2 to 300 amino acid long, and even more preferably 3 to 100 amino acid long. The above vaccine composition may comprise a variety of ingredients for drug formulation such as a pharmaceutically acceptable common carrier, a binder, a stabilizer, an excipient, a diluent, a pH buffer, a disintegrant, a solubilizing agent, a solubilizing aid, and an isotonic agent. The vaccine composition can be administered either orally or parenterally, and for example, the vaccine composition can be orally administered in a dosage form of a powder, a granule, a tablet, a capsule, a syrup, a suspension, and so on, or the vaccine composition prepared in the form of a solution, an emulsion, a suspension, and so on can be parenterally administered by injection. Moreover, the vaccine composition can also be administered to the nasal cavity in a dosage form of a spray agent. However, parenteral administration by injection or drip infusion can be presented as a preferable example.

The vaccine therapy kit of the present invention and the agent for enhancing vaccine therapy effect according to the present invention can also be used for a vaccine therapy with a vaccine composition comprising the steps of a) implanting β-tricalcium phosphate in an inside of a body of a subject; and b) administering a vaccine composition to the subject. The vaccine composition is administered to a subject at the same time with or before or after implantation of β-TCP. The dose and number of doses can be appropriately selected according to the intensity of the activity of the vaccine composition, the type of the disease, the body weight of the patient, the dosage form, and so on, and for example, the daily dose can be preferably 0.0001 to 100 mg/kg (body weight), more preferably 0.01 to 50 mg/kg (body weight), and even more preferably 0.1 to 10 mg/kg (body weight). A preferable example can be such that administration of the vaccine composition is initiated at the same time with or after implantation of the β-TCP of the present invention, and administration is given several times at certain intervals, preferably twice in total with a 2-week interval. Also, the vaccine composition according to the present invention may comprise a complete adjuvant and an incomplete adjuvant, and for example, the vaccine composition of the present invention can be used in combination with a Freund's complete adjuvant or a Freund's incomplete adjuvant. Also, the vaccine composition according to the present invention may be used in combination with administration of medicines other than the vaccine composition, supplements, and so on, or surgical treatment such as surgery.

The agent for enhancing vaccine therapy effect using the vaccine composition according to the present invention has such an effect that when the therapeutic effect achieved by vaccine therapy involving only administration of the vaccine composition is compared with the therapeutic effect achieved by the combined use of a control or the β-TCP composition of the present invention and the vaccine composition, a higher therapeutic effect is achieved when the β-TCP composition is used in combination. The effect of the present invention can be specifically confirmed by examining if the tumor size is reduced or not increased when β-TCP is used in combination with vaccine therapy in comparison with the vaccine therapy alone using, for example, a reduction in the tumor marker level or, for example, the tumor size as an index.

The present invention also has such an effect that a β-TCP-implanted mouse can be used for verification of the effect of a new vaccine composition or for screening for a vaccine composition having a weak efficacy at the stage of a candidate substance. For example, a nude mouse is often used for verification of an anti-tumor effect. Although the only effector cells that may be able to bring about an anti-tumor effect are NK cells in a nude mouse, involvement of not only NK cells, but also T cells and B cells can be studied in a normal mouse in the experimental system according to the present invention. This makes it possible to conduct a study in a setting closer to human clinical conditions (such as cancer patients).

Hereinbelow, the present invention will be further described with reference to specific examples.

### Vaccine Therapy with a Vaccine Composition Using β-TCP

### Specific Example 1

In this experiment, for example, the following experimental groups can be set up.

Group 1: Control group

Group 2: Adjuvant-administered group

Group 3: β-TCP-implanted group

Group 4: Adjuvant-administered, β-TCP-implanted group

Group 5: Adjuvant-OVA peptide-administered group

Group 6: Adjuvant-OVA peptide-administered, β-TCP-implanted group

(1) A discoid β-TCP tablet having a diameter of 5 mm×a thickness of 2 mm (Groups 3, 4, and 6) or a plastic piece of the same size (Groups 1, 2, and 5) are subcutaneously implanted in the flank of a 6-week-old male mouse (C57BL/6).
(2) After 7 days, 1 to 100 μg of ovalbumin (OVA) peptide (amino acid sequence: SIINFEKL) suspended in Freund's complete adjuvant (FCA) or Freund's incomplete adjuvant (FIA) is intradermally administered in the vicinity of the implanted β-TCP tablet or plastic piece.
(3) After 14 days, the aforementioned step (2) is repeated once again.
(4) After 21 days, 1×10⁴ to 1×10⁵ mouse lymphoma cells, i.e., E.G7-OVA (purchased from ATCC), are subcutaneously transplanted in the flank opposite to that in which the implantation and administration have been made, and the formation of tumor and the survival period are observed.

The E.G7-OVA cells used in this experiment are known to express chicken ovalbumin by gene transfer and present OVA peptides on MHC class I. When immunization with OVA peptides effectively works, OVA peptide-specific cytotoxic T cells are induced in the body of the mouse, and even if E.G7-OVA is implanted after immunization, engraftment of tumor is inhibited. Accordingly, when the formation of tumor is suppressed in Group 6 in comparison with Groups 1 to 5, then it can be evaluated that β-TCP has functioned as a vaccine adjuvant.

### Specific Example 2

In this experiment, for example, the following experimental groups can be set up.

Group 1: Control group

Group 2: Adjuvant-administered group

Group 3: β-TCP-implanted group

Group 4: Adjuvant-administered, β-TCP-implanted group

Group 5: Adjuvant-TRP-2 peptide-administered group

Group 6: Adjuvant-TRP-2 peptide-administered, β-TCP-implanted group

(1) A discoid β-TCP tablet having a diameter of 5 mm×a thickness of 2 mm (Groups 3, 4, and 6) or a plastic piece of the same size (Groups 1, 2, and 5) are subcutaneously implanted in the flank of a 6-week-old male mouse (C57BL/6).
(2) After 7 days, 1 to 100 μg of tyrosinase-related protein 2 (TRP-2) peptide (amino acid sequence: VYDFFVWL) suspended in Freund's complete adjuvant (FCA) or Freund's incomplete adjuvant (FIA) is intradermally administered in the vicinity of the implanted β-TCP tablet or plastic piece.
(3) After 14 days, the aforementioned step (2) is repeated once again.
(4) After 21 days, 1×10⁴ to 1×10⁵ mouse melanoma cells, i.e., B16 (purchased from ATCC), are subcutaneously transplanted in the flank opposite to that in which the implantation and administration have been made, and the formation of tumor and the survival period are observed. When the formation of tumor is suppressed in Group 6 in comparison with Groups 1 to 5, then it can be evaluated that β-TCP has functioned as a vaccine adjuvant.

### Specific Example 3

In this experiment, for example, the following experimental groups can be set up.

Group 1: Control group

Group 2: Adjuvant-administered group

Group 3: β-TCP-implanted group

Group 4: Adjuvant-administered, β-TCP-implanted group

Group 5: Adjuvant-WT1 peptide-administered group

Group 6: Adjuvant-WT1 peptide-administered, β-TCP-implanted group

(1) A discoid β-TCP tablet having a diameter of 5 mm×a thickness of 2 mm (Groups 3, 4, and 6) or a plastic piece of the same size (Groups 1, 2, and 5) are subcutaneously implanted in the flank of a 6-week-old male mouse (C57BL/6).
(2) After 7 days, 1 to 100 μg of WT1 peptide (amino acid sequence: RMFPNAPYL) suspended in Freund's complete adjuvant (FCA) or Freund's incomplete adjuvant (FIA) is intradermally administered in the vicinity of the implanted β-TCP tablet or plastic piece.
(3) After 14 days, the aforementioned step (2) is repeated once again.
(4) After 21 days, 1×10⁴ to 1×10⁵ mWT1-C1498 (WT1-expressing murine leukemia cells; purchased from ATCC) is subcutaneously transplanted in the flank opposite to that in which the implantation and administration have been made, and the formation of tumor and the survival period are observed. When the formation of tumor is suppressed in Group 6 in comparison with Groups 1 to 5, then it can be evaluated that β-TCP has functioned as a vaccine adjuvant.

The present invention will be further specifically described with reference to the following Examples; however, the present invention is not limited to Examples.

### EXAMPLES

### Method for Producing β-TCP

Although the β-TCP block can be purchased as a product as an artificial bone, it can also be produced by mixing the raw materials, i.e., β-TCP, water, a surfactant, and a foaming agent, and sintering the resulting mixture. The aforementioned β-TCP block had porous rates of 75% and 60%. Also, in regard to the β-TCP powder and β-TCP granule, a β-TCP powder having a particle diameter of 75 to 105 μm (porous rate: 60 to 70%) was obtained by pulverizing and sieving the aforementioned β-TCP block having a porous rate of 75%. Also, β-TCP of 25 to 75 μm in size (porous rate: 40 to 50%) that was similarly obtained by sieving and a PBS solution were mixed, and immediate after that, the three-fourth of the upper portion of the liquid was collected and subjected to centrifugation at 1000 rpm for 3 minutes. From the precipitate, β-TCP microgranules of 0.05 to 25 μm in size (porous rate: 35 to 50%) were collected, and from the supernatant, microgranules of 0.05 to 5 μm in size (porous rate: 20 to 30%) were collected. Also, a slurry was prepared by mixing calcium hydrogen phosphate, calcium carbonate, and water at an appropriate ratio, and the slurry thus prepared was allowed to undergo reaction while grinding, followed by drying. Subsequently, the solid material thus obtained was pulverized and calcined to give a β-TCP powder. The β-TCP powder thus obtained was formed into tablets having a diameter of 5×2 mm by compression forming and sintered at 600 to 1500° C. for 30 hours, whereby discoid β-TCP dense body tablets were produced (porous rate: 0.1 to 20%). Also, 30 g of the raw material β-TCP powder, 6.0 mL of a surfactant, and 10 mL of water were injected into a centrifugal tube and subjected to defoaming treatment at 250 rpm for 4 minutes, and the resulting product was injected into a mold and dried naturally. After releasing from the mold, the resulting product was sintered at 1300° C., whereby columnar β-TCP dense body bars having a diameter of 0.9×10 mm were produced (porous rate: 10 to 30%). It should be noted that the porous rate of each β-TCP thus produced was measured as a value relative to the sample volume when mercury was forced into a pore having a diameter of approximately 180 μm by a mercury intrusion method, which corresponded to the initial pressure.

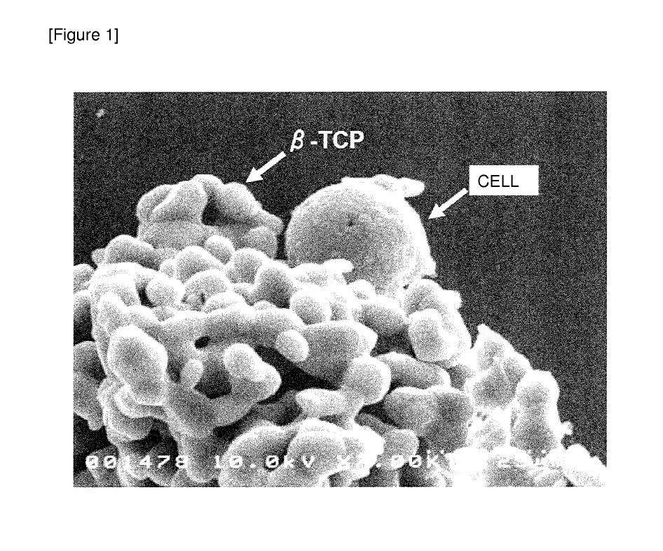

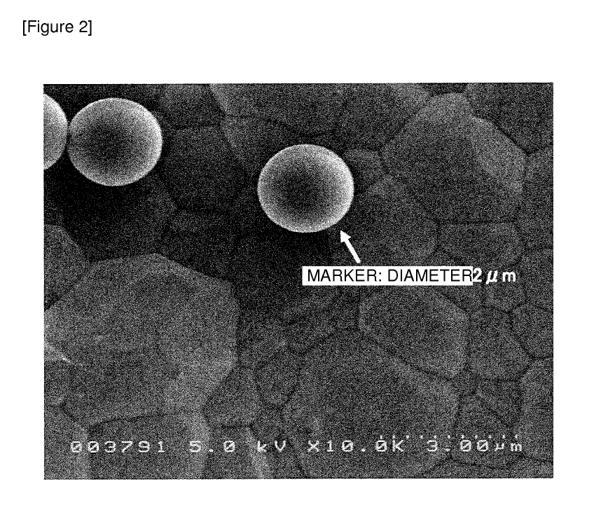

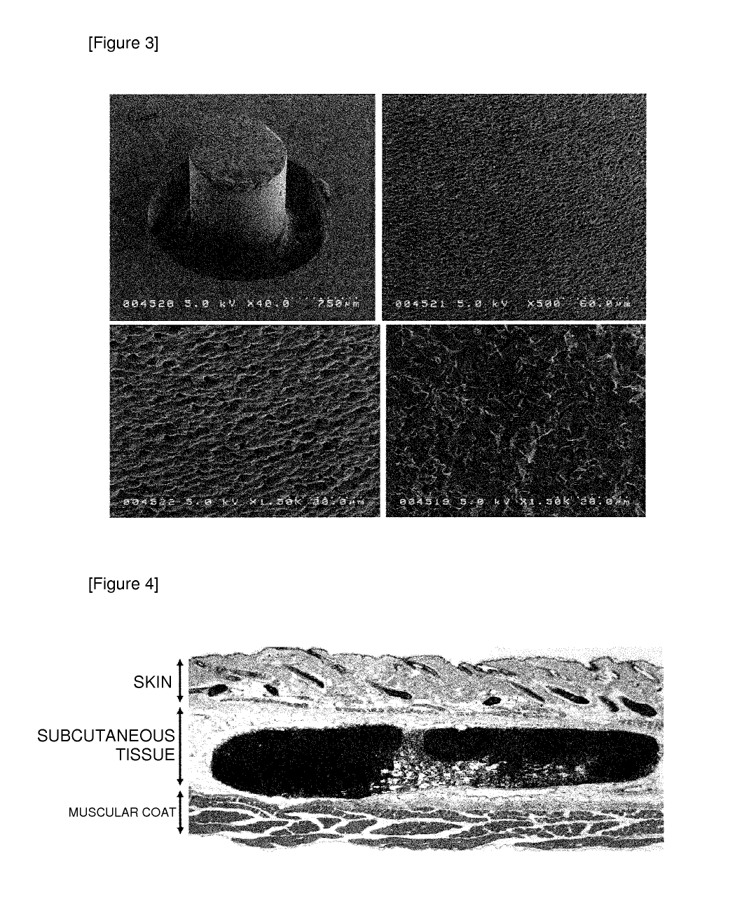

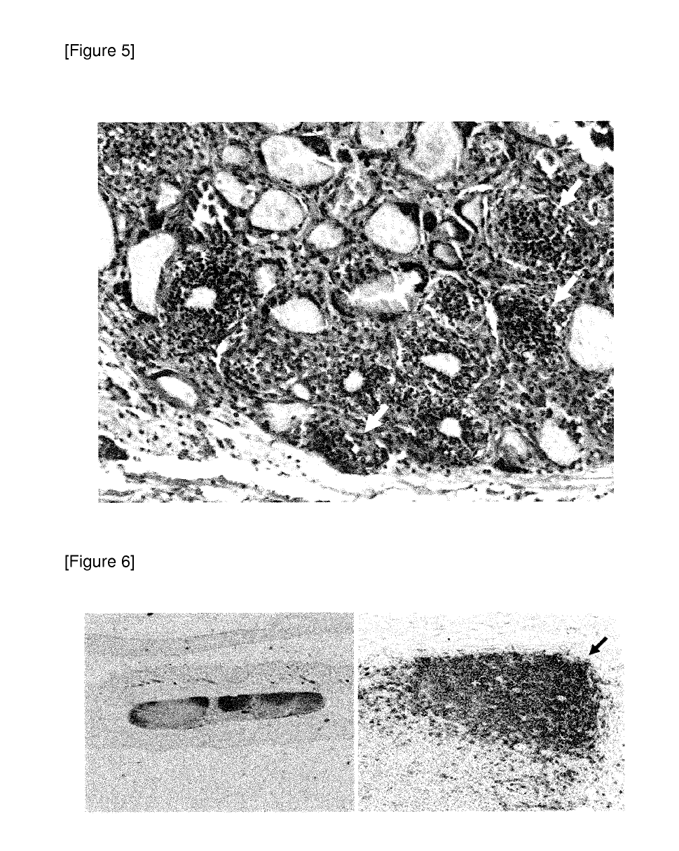

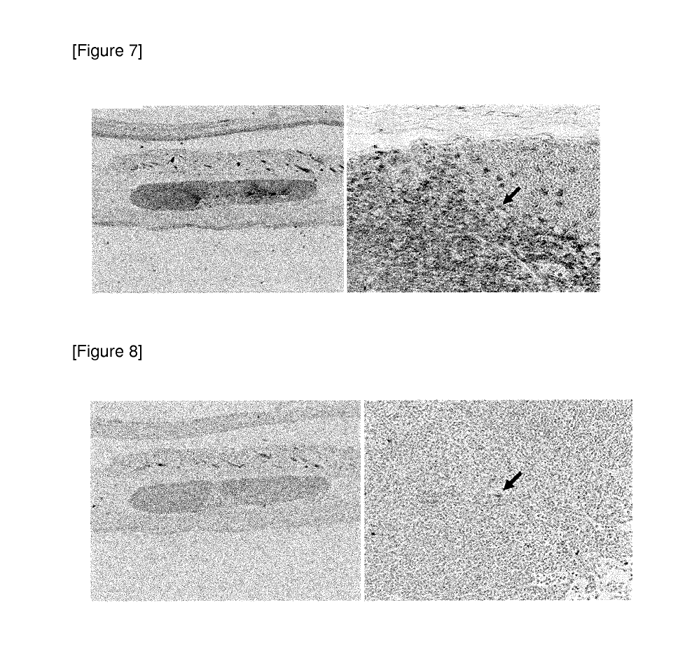

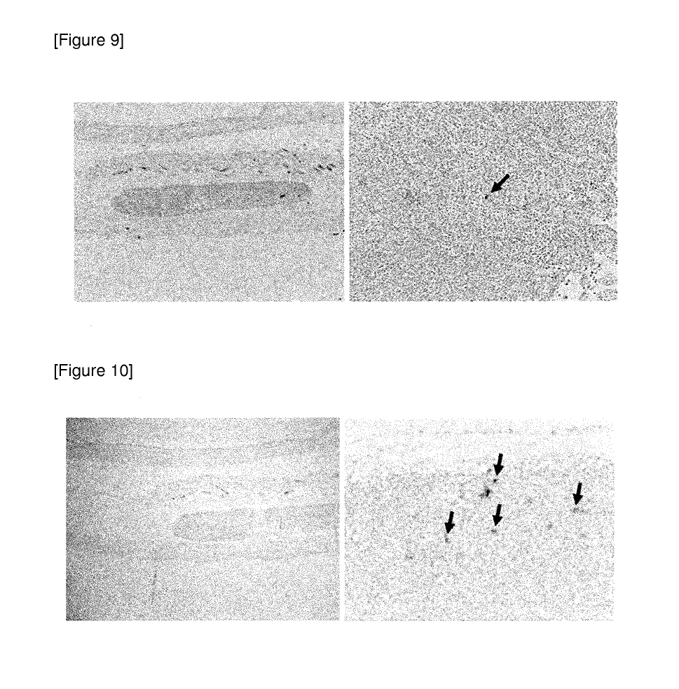

The result of the scanning electron microscopic observation of the binding of the β-TCP granule thus produced (25 to 75 μm in size) to an immune cell is shown in FIG. 1. Also, the results of the scanning electron microscopic observation of the β-TCP dense body tablet and dense body bar thus produced are shown in FIGS. 2 and 3. Further, the result of the microscopic observation of the tissue sample that was collected from a normal mouse subjected to subcutaneous implantation of the β-TCP dense body and was stained with HE is shown in FIG. 4. It was found that lymphocytes accumulated and configured a similar structure to a native lymph node. Also, the result of the microscopic observation of the tissue sample that was collected from a mouse subjected to subcutaneous implantation of the β-TCP granules of 0.05 to 105 μm in size and was stained with HE is shown in FIG. 5. The β-TCP granules of 75 μm or larger in size induced macrophages (N=13), granules of 75 μm or smaller in size gathered lymphocytes (N=11), and microgranules of 0.05 to 25 μm in size formed lymphocyte aggregates. Also, the tissue sample of the mouse that received subcutaneous implantation of the β-TCP dense body having a diameter of 5×2 mm (porous rate: 18%) was immunohistochemically stained with an anti-CD45R antibody, an anti-CD3 antibody, an anti-CD49b antibody, an anti-CD11c antibody, an anti-F4/80R antibody, or an anti-D2-40 antibody for B cells, T cells, NK cells, dendritic cells, macrophages, and lymphatic vessels, respectively, and observed under a microscope. The results are shown in FIGS. 6 to 11. From the above results, it was confirmed that the subcutaneously implanted β-TCP having been densificated by sintering induced lymphocytes (B cells, T cells, and NK cells), dendritic cells, and macrophages, and thus is utilizable as an artificial lymphatic node. A nude mouse is often used for verification of an anti-tumor effect. However, while the only effector cells that may be able to bring about an anti-tumor effect are NK cells in a nude mouse, involvement of T cells and B cells can be studied as well in a mouse into which the β-TCP of the present invention has been implanted. Therefore, it was confirmed that a study was able to be conducted in a setting closer to human clinical conditions (cancer patients).

[Vaccine Therapy with a Vaccine Composition Using β-TCP: No. 1]
(1) As an experimental system of vaccines, a model experimental system of tumor using a C57BL/6 strain mouse and E.G7-OVA (purchased from ATCC), which is the mouse lymphoma cell expressing avian ovalbumin, was used. Using this system, the following 3 groups were set up.

1) Control group: A group of untreated animals in which E.G7-OVA was transplanted.

2) OVA-administered group: A group in which E.G7-OVA was transplanted after administration of OVA protein.

3) OVA+TCP group: A group in which E.G7-OVA was transplanted after implantation of TCP and administration of OVA protein.

A discoid β-TCP dense body (porous rate: 0.1 to 12%) tablet having a diameter of 5 mm×thickness of 2 mm was subcutaneously implanted in the flank of a 6-week-old male mouse (C57BL/6) (OVA+TCP group).

(2) After 7 days, 100 μg of OVA protein was administered intradermally around the implanted β-TCP tablet (OVA+TCP group) or intradermally in the flank (OVA-administered group).
(3) After 14 days, the aforementioned step (2) was repeated once again.
(4) After 21 days, 1×10⁵ E.G7-OVA cells were subcutaneously transplanted in the flank, and the formation and proliferation of tumor were observed.

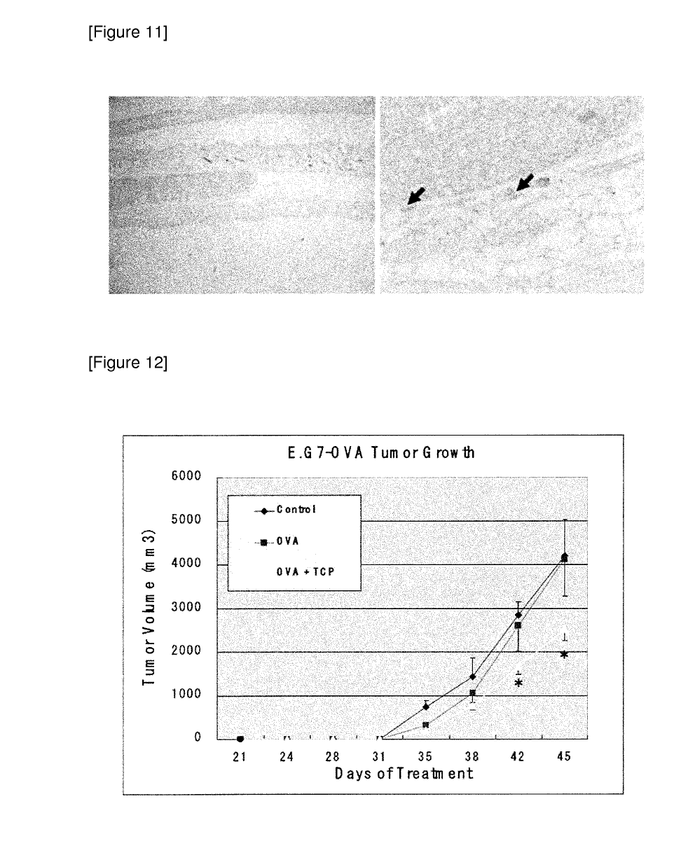

The results are shown in FIG. 12. In FIG. 12, the asterisk * indicates that tumor proliferation was significantly suppressed in the OVA+TCP group, which was immunized with OVA protein after implantation of β-TCP, on 42 days and 45 days after the initiation of the experiment in comparison with the control group (t-test, p<0.05). Given that the E.G7-OVA cells used in this experiment express chicken ovalbumin by gene transfer and present OVA peptides on MHC class I, it is considered that immunization with OVA peptides effectively worked and OVA peptide-specific cytotoxic T cells were induced in the body of the mouse, leading to suppression of tumor proliferation in the group 3).

### INDUSTRIAL APPLICABILITY

The present invention can be favorably used in, for example, the field of medicine such as vaccine therapy, adjunctive therapy for vaccine therapy, and therapeutic method for tumor, and the field of adjuvants, and so on.

1. A method for enhancing a vaccine therapy effect by a vaccine composition, comprising:
a) implanting β-tricalcium phosphate in an inside of a body of a subject, and
b) administering the vaccine composition to the subject at the same time with or before or after step a).

2. The method according to claim 1, wherein the inside of a body is at least one of in a skin, under a skin, and in a muscle.

3. The method according to claim 1, wherein the β-tricalcium phosphate is implanted in a vicinity of a lesion.

4. The method according to claim 3, wherein the vicinity of the lesion is an area at a distance of 0.1 to 15 cm from the lesion.

5. The method according to claim 1, further comprising:
c) administering the vaccine composition 7 days after step b).

6. The method according to claim 1, wherein the vaccine composition is administered at 0.1 to 10 mg/kg body weight.

7. The method according to claim 1, wherein the vaccine composition is a cancer-specific antigen vaccine.

8. The method according to claim 1, wherein the cancer-specific antigen vaccine is at least one of an antigenic peptide and a protein, which are derived from tyrosinase-related protein 2 (TRP-2), Wilms tumor 1 (WT1), ovalbumin (OVA), or a tumor lysate.

9. The method according to claim 1, wherein the β-tricalcium phosphate is in a form of a dense body having a porous rate of 50% or less.

10. The method according to claim 1, wherein the β-tricalcium phosphate is in a form of a dense body having a porous rate of 10 to 30%.

11. The method according to claim 1, wherein the β-tricalcium phosphate activates, induces, or accumulates at least one of T cells, B cells, NK cells, dendritic cells, and macrophages.

12. The method according to claim 1, wherein the β-tricalcium phosphate is in a form of a tablet or a columnar body.

13. The method according to claim 1, wherein the β-tricalcium phosphate is in a form of a particle or granule having a particle diameter of 0.05 to 25 μm.
